(19) United States
(12) Patent Application Publication
(10) Pub. No.: US 2021/0098109 A1
(43) Pub. Date: Apr. 1, 2021

(54) VIRTUAL COMMUNITY SYSTEM FOR ELDERLY HEALTH SERVICE BASED ON BIG DATA TECHNOLOGY

(71) Applicant: Guangzhou Huali Science and Technology Vocational College, Guangzhou (CN)

(72) Inventors: Jianhua WANG, Guangzhou (CN); Jun MA, Guangzhou (CN); Xiaofeng LI, Guangzhou (CN); Wenyu SONG, Guangzhou (CN); Xueling YANG, Guangzhou (CN); Mingyu WU, Guangzhou (CN); Dongcheng GAI, Guangzhou (CN); Guogen FAN, Guangzhou (CN); Zhiyong REN, Guangzhou (CN); Menglin SUN, Guangzhou (CN); Ping LI, Guangzhou (CN); Dan YU, Guangzhou (CN); Yuhua WEI, Guangzhou (CN); Haiquan SONG, Guangzhou (CN); Jun ZHU, Guangzhou (CN)

(21) Appl. No.: 16/906,474

(22) Filed: Jun. 19, 2020

(30) Foreign Application Priority Data
Sep. 27, 2019 (CN) 201910925301.8

(51) Int. Cl. G16H 20/30; G16H 40/67; G16H 10/60; G16H 10/20; G16H 50/70; G16H 70/20; G16H 70/60; G16H 50/20; G16H 20/60; G16H 80/00; G06Q 50/00; G16H 40/20; G06Q 30/02; H04W 4/029; A61B 5/021; A61B 5/00

(52) U.S. Cl. CPC G16H 20/30; H04L 65/403; G16H 10/60; G16H 10/20; G16H 50/70; G16H 70/20; G16H 70/60; G16H 50/20; G16H 20/60; G16H 80/00; G06Q 50/01; G16H 40/20; G06Q 30/0212; H04W 4/029; A61B 5/021; A61B 5/6824; G16H 40/67

(57) ABSTRACT

The present invention discloses a virtual community system for an elderly health service based on a big data technology, including an information obtaining module for obtaining personal information of elderly people, a management platform for processing user information, a recommendation module for sending recommended information to a user, and a backend terminal for providing a manual service. The information obtaining module includes a locator, configured to obtain the elderly people's position information to prevent the elderly people from getting lost; and a health bracelet, configured to detect the elderly people's heart rate and blood pressure which directly reflect the elderly people's physical condition. The present invention is designed to meet an existing need. One-time treatment for a lifetime medical service is implemented based on existing medical data and personal data acquired by health equipment.

### TECHNICAL FIELD

The present invention relates to the field of elderly health management system technologies, and in particular, to a virtual community system for an elderly health service based on a big data technology.

### BACKGROUND

With the development of Internet technologies and the advent of the era of big data, all industries are facing industrial service upgrades, and users urgently need more comprehensive and thoughtful medical and health services. In this subject, a big data technology is used to upgrade and reform an existing medical service model, a one-stop personalized health management service platform is established for patients, and a solution for integrating multidimensional heterogeneous data is proposed based on existing medical data and personal data acquired by health equipment. In addition, a detailed solution for constructing a health management service model based on intelligent cloud collaboration is proposed based on integrated multidimensional heterogeneous data in combination with a big data algorithm and a cloud computing technology. In this way, one-time treatment for a lifetime medical service is implemented. An existing medical service process is reformed to adapt to development of the era of artificial intelligence, and provide convenient and intelligent medical treatment for patients.

To cater to the above need, a virtual community system for an elderly health service based on a big data technology is provided.

### SUMMARY

The objective of the present invention is to provide a virtual community system for an elderly health service based on a big data technology, to solve the problem raised in the background.

To achieve the above objective, the present invention provides the following technical solution.

A virtual community system for an elderly health service based on a big data technology is disclosed, including an information obtaining module for obtaining personal information of elderly people, a management platform for processing user information, a recommendation module for sending recommended information to a user, and a backend terminal for providing a manual service.

The information obtaining module includes a locator, configured to obtain position information of the elderly people to prevent the elderly people from getting lost;

a health bracelet, configured to detect the elderly people's heart rate and blood pressure which directly reflect the elderly people's physical condition; and

a registration unit, configured to obtain specific identity information of the elderly people, including age, height, weight, regular diet, contact information, electronic physical examination form, and address information, wherein the contact information includes contact information of the elderly people and that of the elderly people's family member.

The information obtaining module further includes a pedometer, configured to obtain the elderly people's usual exercise status.

The management platform includes a database and a processor. The processor is configured to perform data matching based on the elderly people's physical condition obtained by the information obtaining module to evaluate the elderly people's physical condition. The database is configured to store related symptoms and treatment methods of various diseases.

The recommendation module includes a dietary recommendation unit, configured to recommend a menu suitable for the elderly people based on the elderly people's information processed by the management platform, so that the elderly people can supplement necessary nutrition from diet; and

an exercise recommendation unit, configured to recommend the amount of daily or monthly exercise to the elderly people, for example, walking 10,000 steps a day; and recommend an exercise type based on the elderly people's physical condition, for example, recommend walking or practicing Tai Chi for elderly people having a weak constitution, and recommend aerobic exercise such as jogging for elderly people having a good constitution.

The backend terminal comprises a controller for wireless interaction with the elderly people or the elderly people's family member. An operator monitors the controller to urge elderly people who cannot improve by themselves, so that the elderly people can develop a good healing habit with the help of an external force.

The recommendation module further includes a case recommendation unit, configured to recommend knowledge corresponding to the elderly people's physical condition to the elderly people.

The case recommendation unit is further configured to recommend information about a related cured patient to the elderly people to enhance confidence of the elderly people through communication between the cured patient and the elderly people.

The recommendation module further includes a group chat recommendation unit, configured to construct a plurality of groups themed on different interests in the form of QQ groups or WeChat groups to facilitate spiritual communication between elderly people.

The recommendation module further includes a medical recommendation unit, configured to recommend a corresponding hospital and a doctor in charge of the disease in the hospital based on the elderly people's physical condition, including personal information of the doctor.

The exercise recommendation unit is further configured to collect statistics on exercise statuses of registered elderly people, obtain a quantity A of exercise steps of each elderly people, construct a discount draw system, and set a draw condition. Only elderly people whose quantity A of exercise steps is greater than a designated value can participate in the draw system. The draw system provides a daily necessity discount to attract the elderly people to exercise, thereby ensuring physical health and improving a happiness index of exercise.

The exercise recommendation unit further includes a plurality of checkpoints set up on different road sections. Punching in can be performed through induction, APP code scanning, or fingerprints. A plurality of checkpoints are set up in exercise places such as a community park for the elderly. An exercise requirement of the system on elderly people is met only when the elderly people punches in at every checkpoint. When the exercise requirement set in the system is met, the elderly people can participate in a draw.

Compared with the prior art, the beneficial effects of the present invention are as follows: The present invention is designed to meet an existing need. A solution for integrating multidimensional heterogeneous data is proposed based on existing medical data and personal data acquired by health equipment. In addition, a detailed solution for constructing a health management service model based on intelligent cloud collaboration is proposed based on integrated multidimensional heterogeneous data in combination with a big data algorithm and a cloud computing technology. In this way, one-time treatment for a lifetime medical service is implemented. In this project, an existing medical service process is reformed to better adapt to development of the era of artificial intelligence, and provide convenient and intelligent medical treatment for patients.

### BRIEF DESCRIPTION OF DRAWINGS

FIG. 1 is a schematic structural diagram of the present invention;

FIG. 2 is a schematic structural diagram of an information obtaining module according to the present invention; and

FIG. 3 is a schematic structural diagram of a recommendation module according to the present invention.

### DETAILED DESCRIPTION

The following clearly and completely describes the technical solutions in the examples of the present invention with reference to accompanying drawings in the examples of the present invention. Apparently, the described examples are merely a part rather than all of the examples of the present invention. All other examples obtained by a person of ordinary skill in the art based on the examples of the present invention without creative efforts shall fall within the protection scope of the present invention.

### Example 1

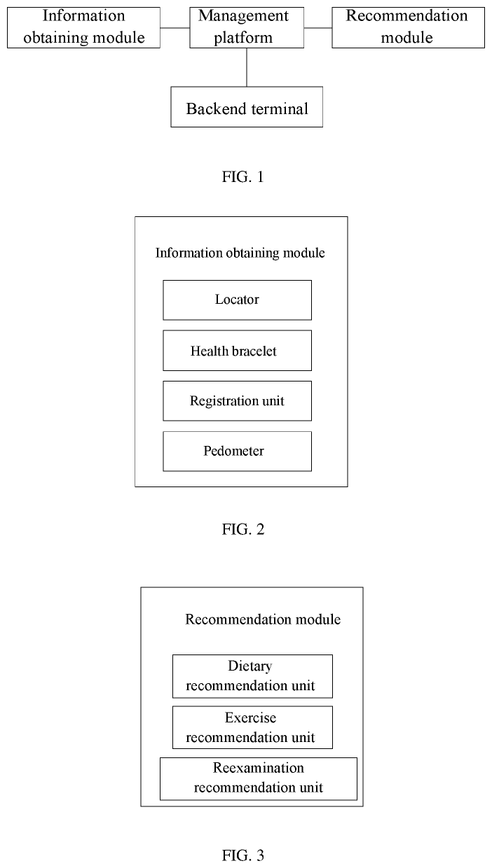

Referring to FIG. 1 to FIG. 3, in this example of the present invention, a virtual community system for an elderly health service based on a big data technology includes an information obtaining module for obtaining personal information of elderly people, a management platform for processing user information, a recommendation module for sending recommended information to a user, and a backend terminal for providing a manual service.

The information obtaining module includes a locator, configured to obtain the elderly people's position information to prevent the elderly people from getting lost;

a health bracelet, configured to detect the elderly people's heart rate and blood pressure which directly reflect the elderly people's physical condition; and

a registration unit, configured to obtain specific identity information of the elderly people, comprising age, height, weight, regular diet, contact information, electronic physical examination form, and address information, where the contact information includes contact information of the elderly people and that of the elderly people's family member.

The information obtaining module further includes a pedometer, configured to obtain the elderly people's usual exercise status.

The management platform includes a database and a processor. The processor is configured to perform data matching based on the elderly people's physical condition obtained by the information obtaining module to evaluate the elderly people's physical condition. The database is configured to store related symptoms and treatment methods of various diseases.

The recommendation module includes a dietary recommendation unit, configured to recommend a menu suitable for the elderly people based on the elderly people's information processed by the management platform, so that the elderly people can supplement necessary nutrition from diet;

an exercise recommendation unit, configured to recommend the amount of daily or monthly exercise to the elderly people, for example, walking 10,000 steps a day; and recommend an exercise type based on the elderly people's physical condition, for example, recommend walking or practicing Tai Chi for elderly people having a weak constitution, and recommend aerobic exercise such as jogging for elderly people having a good constitution; and

a reexamination recommendation unit, configured to recommend a reexamination cycle and a reexamination focus to the elderly people based on the elderly people's physical information. The recommended reexamination cycle helps the elderly people regularly maintain the elderly people's body, get positive feedback, and stick to a good habit. The reexamination focus helps the elderly people focus on a certain aspect of the body during reexamination.

The backend terminal includes a controller for wireless interaction with the elderly people or the elderly people's family member. An operator monitors the controller to urge elderly people who cannot improve by themselves, so that the elderly people can develop a good healing habit with the help of an external force. The operator can also contact the elderly people's family member by using the controller to assist the elderly people in completing treatment with the help of the family member.

### Example 2

The recommendation module further includes a case recommendation unit. The case recommendation unit is configured to recommend knowledge corresponding to the elderly people's physical condition to the elderly people, for example, recommend advantages and disadvantages of being thin to a thin elderly people, and

provide precautions about high blood pressure and a method for lowering blood pressure for elderly people with high blood pressure.

The case recommendation unit is further configured to recommend information about a related cured patient to the elderly people to enhance confidence of the elderly people through communication between the cured patient and the elderly people.

### Example 3

The recommendation module further includes a group chat recommendation unit, configured to construct a plurality of groups themed on different interests in the form of QQ groups or WeChat groups. Elderly people with the same hobby or disease are added to one group. In this case, the elderly people will not feel lonely when their children are not around them, thereby meeting the elderly people′ spiritual needs.

### Example 4

The recommendation module further includes a medical recommendation unit. The medical recommendation unit is configured to recommend a corresponding hospital and a doctor in charge of treatment for the disease in the hospital based on the elderly people's physical condition, including personal information of the doctor. This prevents the elderly people from blindly seeking medical treatment.

### Example 5

The exercise recommendation unit is further configured to collect statistics on exercise statuses of registered elderly people, obtain a quantity A of exercise steps of each elderly people, construct a discount draw system, and set a draw condition. Only elderly people whose quantity A of exercise steps is greater than a designated value can participate in the draw system. The draw system provides a daily necessity discount to attract the elderly people to exercise, thereby ensuring physical health and improving a happiness index of exercise. In this case, it is easier for the elderly people to develop an exercise habit. Nowadays, it is difficult for people to develop the exercise habit because an exercise effect is invisible and no feedback is given for the people's effort in a short period of time.

### Example 6

The exercise recommendation unit further includes a plurality of checkpoints set up on different road sections. Punching in can be performed through induction, APP code scanning, or fingerprints. A plurality of checkpoints are set up in exercise places such as a community park for the elderly. An exercise requirement of the system on elderly people is met only when the elderly people punches in at every checkpoint. When the exercise requirement set in the system is met, the elderly people can participate in a draw. This helps the elderly people to develop the habit of punching in at fixed points, so that the elderly people naturally gets into the habit of exercising every day.

A working principle of the present invention is as follows: In the present invention, the identity information and pathological information of the elderly people are acquired. The management platform analyzes and evaluates the elderly people's physical condition. Then the recommendation module recommends the corresponding exercise intensity, exercise method, diet, and reexamination cycle to the elderly people. The elderly people can learn the specific physical condition of the elderly people and the corresponding knowledge to avoid the fear of illness. The operator can communicate with the elderly people by using the backend terminal to assist treatment for the elderly people with the external force. Further, a detailed solution for constructing a health management service model based on intelligent cloud collaboration is formed to implement one-time treatment for a lifetime medical service.

It apparent for those skilled in the art that the present invention is not limited to the details of the above exemplary examples, and that the present invention may be implemented in other specific forms without departing from the spirit or basic features of the present invention. The examples should be regarded as exemplary and non-limiting in every respect, and the scope of the present invention is defined by the appended claims rather than the above description. Therefore, all changes falling within the meaning and scope of equivalent elements of the claims should be included in the present invention. Any reference signs in the claims should not be considered as limiting the claims involved.

It should be understood that although this specification is described in accordance with the examples, not every example includes only an independent technical solution. Such a description is merely for the sake of clarity, and those skilled in the art should take the specification as a whole. The technical solutions in the examples can also be appropriately combined to form other implementations which are comprehensible for those skilled in the art.

What is claimed is:

1. A virtual community system for an elderly health service based on a big data technology, comprising an information obtaining module for obtaining personal information of elderly people, a management platform for processing user information, a recommendation module for sending recommended information to a user, and a backend terminal for providing a manual service, wherein
the information obtaining module comprises a locator, configured to obtain the elderly people's position information to prevent the elderly people from getting lost;
a health bracelet, configured to detect the elderly people's heart rate and blood pressure which directly reflect the elderly people's physical condition; and
a registration unit, configured to obtain specific identity information of the elderly people, such as age, height, weight, regular diet, contact information, electronic physical examination form, and address information, wherein the contact information comprises contact information of the elderly people and that of the elderly people's family member;
the management platform comprises a database and a processor, wherein the processor is configured to perform data matching based on the elderly people's physical condition obtained by the information obtaining module to evaluate the elderly people's physical condition, and the database is configured to store related symptoms and treatment methods of various diseases;
the recommendation module comprises a dietary recommendation unit, configured to recommend a menu suitable for the elderly people based on the elderly people's information processed by the management platform, so that the elderly people can supplement necessary nutrition from diet; and
an exercise recommendation unit, configured to recommend the amount of daily or monthly exercise to the elderly people, for example, walking 10,000 steps a day; and recommend an exercise type based on the elderly people's physical condition, for example, recommend walking or practicing Tai Chi for elderly people having a weak constitution, and recommend aerobic exercise such as jogging for elderly people having a good constitution; and
the backend terminal comprises a controller for wireless interaction with the elderly people or the elderly people's family member, wherein an operator monitors the controller to urge elderly people who cannot improve by themselves, so that the elderly people can develop a good healing habit with the help of an external force.

2. The virtual community system for an elderly health service based on a big data technology according to claim 1, wherein the recommendation module further comprises a reexamination recommendation unit, configured to recommend a reexamination cycle and a reexamination focus to the elderly people based on the elderly people's physical information, wherein the recommended reexamination cycle helps the elderly people regularly maintain the elderly people's body, get positive feedback, and stick to a good habit, and the reexamination focus helps the elderly people focus on a certain aspect of the body during reexamination.

3. The virtual community system for an elderly health service based on a big data technology according to claim 1, wherein the recommendation module further comprises a case recommendation unit, configured to recommend knowledge corresponding to the elderly people's physical condition to the elderly people.

4. The virtual community system for an elderly health service based on a big data technology according to claim 3, wherein the case recommendation unit is further configured to recommend information about a related cured patient to the elderly people to enhance confidence of the elderly people through communication between the cured patient and the elderly people.

5. The virtual community system for an elderly health service based on a big data technology according to claim 1, wherein the recommendation module further comprises a group chat recommendation unit, configured to construct a plurality of groups themed on different interests in the form of QQ groups or WeChat groups to facilitate spiritual communication between elderly people.

6. The virtual community system for an elderly health service based on a big data technology according to claim 1, wherein the recommendation module further comprises a medical recommendation unit, configured to recommend a corresponding hospital and a doctor in charge of the disease in the hospital based on the elderly people's physical condition, comprising personal information of the doctor.

7. The virtual community system for an elderly health service based on a big data technology according to claim 1, wherein the registration unit further comprises a pedometer, configured to obtain the elderly people's usual exercise status.

8. The virtual community system for an elderly health service based on a big data technology according to claim 1, wherein the exercise recommendation unit is further configured to collect statistics on exercise statuses of registered elderly people, obtain a quantity A of exercise steps of each elderly people, construct a discount draw system, and set a draw condition, wherein only elderly people whose quantity A of exercise steps is greater than a designated value can participate in the draw system, and the draw system provides a daily necessity discount to attract the elderly people to exercise, thereby ensuring physical health and improving a happiness index of exercise.

9. The virtual community system for an elderly health service based on a big data technology according to claim 8, wherein the exercise recommendation unit further comprises a plurality of checkpoints set up on different road sections; punching in can be performed through induction, APP code scanning, or fingerprints; a plurality of checkpoints are set up in exercise places such as a community park for the elderly; an exercise requirement of the system on elderly people is met only when the elderly people punches in at every checkpoint; and when the exercise requirement set in the system is met, the elderly people can participate in a draw.
